(19) United States
(12) Patent Application Publication
(10) Pub. No.: US 2008/0125765 A1
(43) Pub. Date: May 29, 2008

(54) Microwave apparatus for ablation

(76) Inventors: A. Berenshteyn, Ocean, NJ (US); G. Kleyman, Brooklyn, NY (US)

Correspondence Address: The Law Firm of Y. Kateshov, 174 Ferndale Road, SCARSDALE, NY, 10583, US

(21) Appl. No.: 11/603,866

(22) Filed: Nov. 24, 2006

(51) Int. Cl. A61B 18/18

(52) U.S. Cl. 606/33

(57) ABSTRACT

An apparatus for ablating biological tissues is configured with a cannula, a balloon inflatable with a gaseous medium and coupled to the cannula, and a microwave antenna in the balloon operative to emit radio waves which heat the peripheral wall of the balloon. The peripheral wall is made from wave penetrating material impregnated with a plurality of wave absorbing particle which are heated to the desired ablation temperature by the absorbed radio waves.

### BACKGROUND OF INVENTION

1. Field of the Invention

The invention relates to a microwave-based apparatus for ablating biological tissues.

2. Prior Art

There are known medical devices in the prior art used for thermal ablation of diseased biological tissues which are operative to apply heat, either directly or indirectly, to such tissues. It is also well known to utilize at least some of the known devices with inflatable balloons inserted into a patient's cavity.

The known devices for ablating biological tissue typically utilize a liquid to inflate the balloon after the device is inserted into a cavity for treatment. The liquid is then heated to a certain temperature and for a period of time sufficient to cause the ablation of tissue. Accordingly, liquids function as a heat capacitor. Such known devices are configured to prevent generating heat above the boiling temperature. Typically, liquids used for the discussed apparatus reach the boiling point at temperatures somewhat higher than 70° C. for water or water-based solutions and 195° C. for Glycerin. Heating the liquid around the boiling point causes gasification of the liquid in the balloon and, as a result, uneven distribution of heat transferred through the balloon's periphery, since gases and liquids have different rates of thermal conductivity. As a result, a region or regions of deceased tissue may be inadequately ablated, while healthy tissues may be detrimentally heated. Clearly, utilizing liquids as a heat-conductive element in an ablation apparatus is associated with undesirable heat-distribution effects that may lead to serious health complications or inadequately performed surgeries.

Furthermore, the known devices are often configured with a low frequency power source (less than 300 MHZ) typically heating the liquid at relatively low temperatures. As a consequence, the use of low radio frequency power sources requires a prolonged time period to generate the sufficient amount of heat produced by the liquid and causing the ablation. During that heat exposure time, the heat transfers from treated diseased tissues to neighboring healthy tissues and may damage the latter. Therefore, the use of liquids in ablation devices is associated with a few health-related problems requiring a comprehensive solution.

It is not unusual for an inflatable balloon to get ruptured. The thermal capacity of a liquid in the balloon is relatively large. If a relatively hot liquid is inadvertently released from the balloon into a cavity, not only it may damage the outer layer of healthy tissues, but it also may penetrate at a substantial depth into the inner layers of tissues which underlie both the healthy and deceased outer tissue layers. As a consequence, the balloon inflatable by a liquid may present health problems.

Also, the regions of deceased tissue to be ablated are typically localized and, thus, relatively small compared to the entire area of healthy biological tissue which is juxtaposed with an inflatable balloon. Consequently, heating the entire periphery of the balloon is usually unnecessary and, again, may be hazardous to a large region of healthy tissue. A need therefore exists in configuring the balloon with selectively heatable peripheral regions to target the regions of deceased tissue while minimizing heating the healthy tissue.

It is, therefore, desirable to provide an apparatus for thermally treating a biological tissue that allows for a relatively brief treatment in a safe and target-oriented manner.

It is also desirable to provide an apparatus for thermally treating a biological tissue by utilizing a gaseous medium as thermally conductive fluid filling a balloon.

It is further desirable to provide an apparatus for thermally treating a biological tissue that is powered by a microwave source to minimize a period of time necessary for reaching the desirable temperature.

It is still further desirable to provide an apparatus for thermally treating a biological tissue that has an inflatable balloon configured with selective thermo-conducting areas to target deceased tissues while minimizing heat exposure of healthy tissues.

### SUMMARY OF THE INVENTION

These needs are satisfied by the inventive apparatus for ablation operable for selectively heating a biological tissue in a cavity so as to minimize exposure of a healthy tissue to heat. The apparatus is configured with a cannula provided with a body which is shaped and dimensioned to penetrate a cavity in a body of a patient and with a heat-conductive component—inflatable balloon—coupled to the body and configured to thermally treat a deceased tissue in the cavity. The apparatus further has an antenna coupled to the cannula and exitable to radiate electromagnetic waves in a microwave range which propagate through fluid in the balloon.

According to one aspect, the inventive apparatus operates with a gaseous medium filling the inflatable balloon and with a microwave power source. The use of the gaseous medium and microwave energy accelerates heating at least a portion of the balloon's peripheral wall, which is impregnated with particle filers, and leaves the low density gaseous medium practically thermally unaffected. As a result, the risk of thermally damaging the biological tissue, if and when the balloon is ruptured or leaks, considerably minimized. In contrast, of course, if the balloon was filled with liquid, as disclosed in the known prior art devices, heat would be absorbed by the latter and, if the balloon ruptures, the heated liquid may damage a large, deep region of biological tissue.

In accordance with a further aspect of the invention, the peripheral wall of the balloon is configured to be selectively heated to a predetermined temperature for thermally treating the deceased tissue, while neighboring regions of the peripheral wall remain unheated. This is achieved by providing the peripheral wall of the balloon, which allows radio waves to penetrate therethrough, with at least one wall region in which wave penetrating material is impregnated with wave absorbing particles or fillers. Generating radio waves in a frequency range, which is roughly up to 3000 megahertz (3 gigahertz), the wave absorbing particles absorb microwave energy which is, thus, transferred into heat energy. At the same time, the regions of the peripheral wall which are free from the heat absorbing particles remain substantially thermally unaffected. As a result, upon inserting the balloon into a cavity, the heat absorbing region or regions of the balloon juxtaposed with deceased tissues provide effective thermal treatment of the targeted deceased tissues. The above and other features and advantages of the disclosed apparatus will be described hereinbelow in conjunction with the following drawings.

### BRIEF DESCRIPTION OF THE DRAWINGS

FIG. 1 is a view of the inventive thermal ablation apparatus configured with a cannula, a microwave oscillator in electrical communication with the handpiece, and a control system operative to monitor and control fluid pressure in a balloon and the temperature of the balloon;

FIG. 2 is a cross-sectional view of the handpiece of FIG. 1;

FIG. 3 is a side elevational view of the inflatable balloon of FIG. 2 configured in accordance with one embodiment of the invention;

FIG. 4 is a side elevational view of the balloon of FIG. 2 configured is accordance with another embodiment of the invention;

FIG. 5 is a side elevational view of the apparatus of FIG. 2 having a cannula and an antenna configured in accordance with a further embodiment of the invention;

FIG. 6 is a side elevational view of the apparatus of FIG. 5 illustrating a further embodiment of the invention;

FIG. 7 is an enlarged cross-sectional view of an inlet fluid port provided in the handpiece of FIG. 2 and in flow communication with the fluid supply system of FIG. 1;

FIG. 8 is an enlarged cross-sectional view of an outlet fluid port of the handpiece of FIG. 2 in flow communication with the inlet port and opening into the inflatable balloon; and

FIG. 9 is a schematic view of power and fluid supply and control systems.

### DETAILED DESCRIPTION

Reference will now be made in detail to several views of the invention that are illustrated in the accompanying drawings. Wherever possible, same or similar reference numerals are used in the drawings and the description to refer to the same or like parts or steps.

The drawings are in simplified form and are not to precise scale. For purposes of convenience and clarity only, directional terms, such as rear and front may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

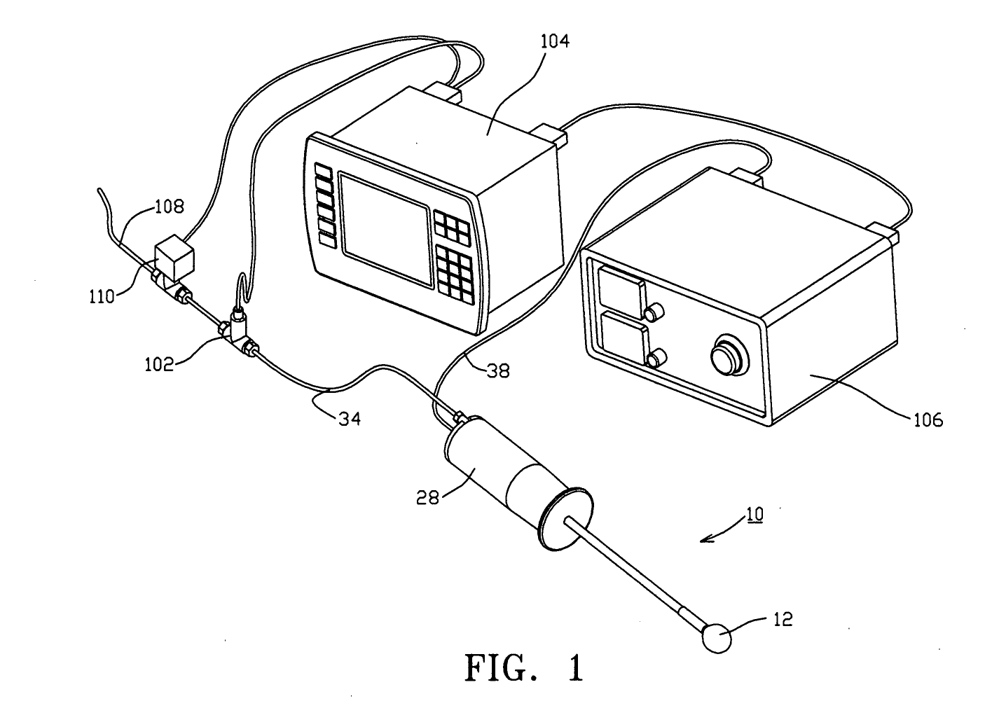

FIG. 1 illustrates an overall view of a microwave apparatus for ablation configured in accordance with the invention and operative to perform a minimally invasive surgery associated with a thermal treatment of biological tissues in general and, in particular, endometrial ablation. A cannula 10, shaped and dimensioned to be introduced into a cavity, includes an inflatable balloon 12 operatively supported by cannula 10. The balloon 12 receives pressurized fluid, such as air or other gaseous medium, through a pneumatic supply line 34 and expands to the desired position. A microwave generator or oscillator 106 is coupled to an antenna 14 located within balloon 12 by means of conductive elements or wires 38. When excited, antenna 14 emits microwaves that propagate through the gaseous medium and are selectively absorbed by the peripheral wall of balloon 12 so that wave absorbing wall regions are heated, whereas wave penetrating wall regions remain substantially thermally unaffected. The temperature and pressure control of fluid are monitored by a control unit 104 operating a pressure transducer 110 and a valve 102 in a manner discussed hereinbelow. The use of gaseous medium heated by microwave generator 106 provides for rapid heating of the wave absorbing regions of balloon 12, effective ablation of the deceased tissue and a time-effective, safe operation, since fluid practically does not absorb microwaves.

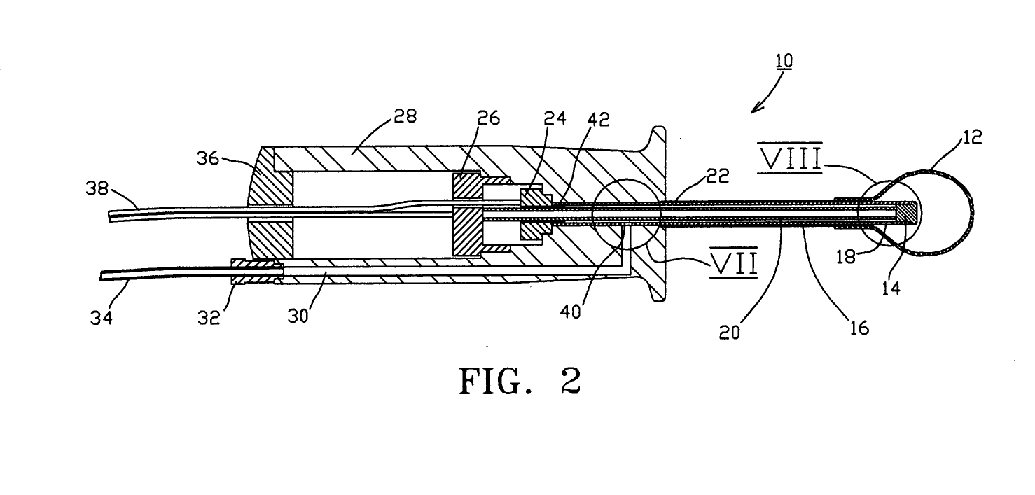

Referring to FIG. 2, cannula 10 is configured with an elongated body 28 made from a heat-insulating material, such as a plastic. The rear or proximal end of body 28 has a cavity closable by a plug 36 which is traversed by wires 38. The wires 38 are coupled to respective elements 26 and 24 which are mounted to the inner surface of body 28 and spaced from plug 36. The elements 24 and 26 are electrically isolated relative to one another and further electrically coupled to respective outer and inner electrodes 16 and 20 which are surrounded by a shield 22 made from heat-shrinking material and circumferentially spaced from one another. The body 28 is provided with distal element 24 and has its distal end sealed to the open end of balloon 12. The outer or distal ends of respective electrodes 16 and 20 are bridged by a microwave antenna 14 locating within inflatable balloon 12 and operative to emit radio waves propagating in a gaseous medium within balloon 12. The balloon 12 is made from elastomer, which, for example, can be silicon. Silicones are generally unaffected by exposure to temperatures reaching 500° F. As a result, those wall regions of balloon 12 that contain only silicone remain substantially unheated and do not detrimentally affect the surrounding biological tissue upon exiting antenna 14.

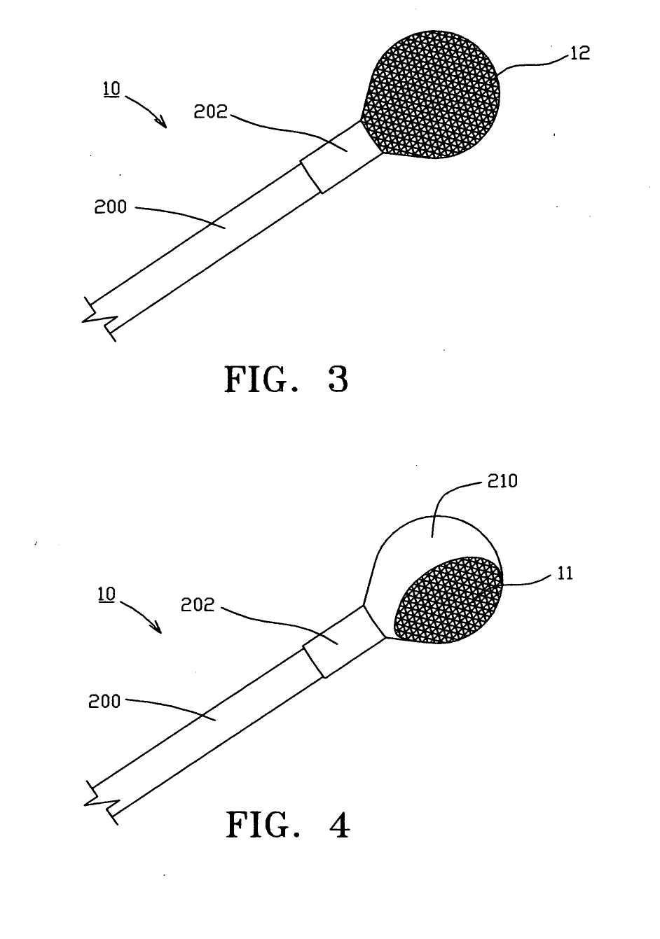

As illustrated in FIG. 3, balloon 12 is attached to a sleeve 202 of cannula 200. To provide heated regions on the peripheral wall of the balloon, it is filled with wave-absorbed particles including but not limited to nickel, nickel-plated graphite, silver-plated aluminum, silver-plated copper, silver-plated nickel, silver-plated glass, pure silver, fluorosilicone, fluorocarbon, and ethylene-propylene terpolymer (EPDM). In the embodiment of FIG. 3, these particles are distributed over the entire peripheral wall of balloon 12. Thus, radio frequency waves emitted by antenna 14 propagating through the gaseous medium and further through portions of balloon 12 free from wave-absorbed particles do not substantially thermally affect both. However, impinging upon the particles, EM energy is transferred into heat energy manifested by heat which is produced by the particles.

Frequently, the tissue to be treated is rather small compared to the entire periphery of balloon 12. Accordingly, providing the peripheral wall of balloon 12 with a target oriented wave absorbing region may be beneficial to the patient's health and allow for a time-effective surgery.

As shown in FIG. 4, the peripheral wall of balloon 12 has one or more heatable regions 11, which include polymeric material impregnated with wave-absorbing particles elements, and peripheral regions that do not have wave-absorbing elements. The heatable regions 11 can be patterned so that when cannula 10 is inserted into the cavity, these regions will be juxtaposed with regions of deceased biological tissue. A microwave antenna in balloon 12 is centered on the longitudinal axis of balloon 12, as shown in FIG. 2, and emits radio frequency waves. The microwaves propagate through a gaseous medium and further penetrate electro-conductive elements of region or regions 11 enabling, thus, a rapid and high-intensity heat transfer therethrough. On the other hand, the rest of balloon's peripheral wall that does not have filers remains thermally unaffected by penetrating microwaves and does not affect a healthy biological tissue, which is juxtaposed with the filler-free peripheral wall regions. The region 11 may be variously shaped, dimensioned and located in accordance with target areas containing deceased biological tissues upon inserting cannula 200 into the cavity. In addition to target configured region or regions 11, balloon 12 may be variously shaped and dimensioned to address specific needs of any given patient.

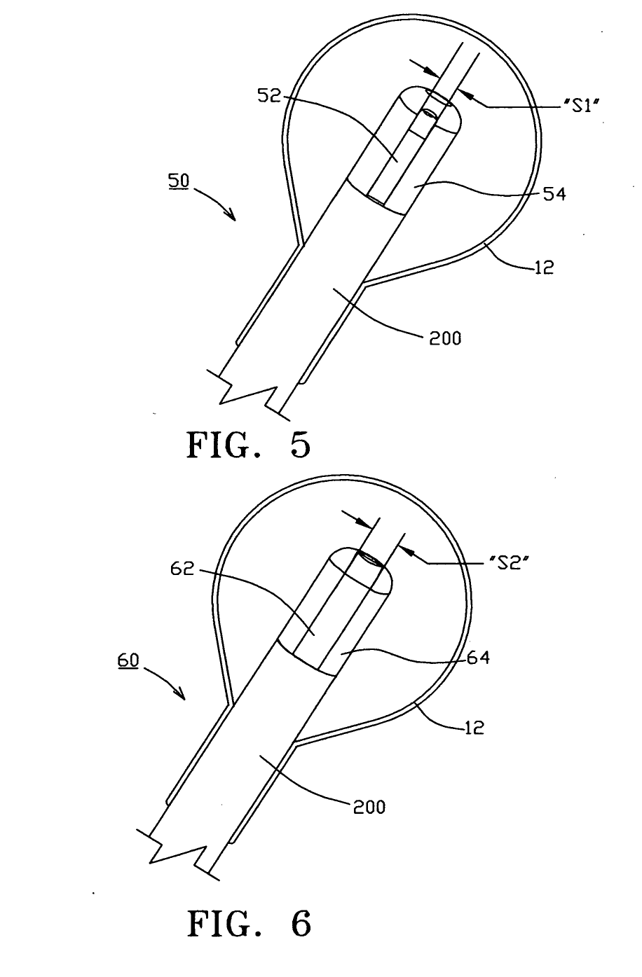

FIG. 5 illustrates a further embodiment of inventive apparatus 50 provided with a cannula 200 which is configured to localize microwave heating of the balloon's periphery. The distal end 54 of cannula 200 has an elongated channel extending generally coaxially with the longitudinal axis of cannula 200 and opening into the distal tip of cannula 200. The channel is shaped and dimensioned to receive a microwave antenna 52 having its distal end spaced inwards from the open tip of cannula 200. As a consequence, when antenna 52 is exited, the waves generally extend along a predetermined path S1, defined by the opening in the tip of the cannula channel, and heat the desired region of balloon 12 which is juxtaposed with a deceased tissue in the cavity. Although the channel and antenna 52 are shown to be centered about the longitudinal axis of cannula 200, other modifications of the shape of the channel may include bent regions. For example, the channel may have a distal end 53 extending transversely to the longitudinal axis of cannula 200, as shown by dash lines in FIG. 5, and opening into a respective side opening of distal end 54 of cannula 200. The antenna 52 also has its distal end extending transversely to the longitudinal axis along the distal end of the channel. Furthermore, the configuration of the channel may include multiple transverse passages and each having a respective portion of antenna 52.

FIG. 6 illustrates a further modification of inventive apparatus 60 configured with cannula 200 having its distal end 64 machined so as to receive a microwave antenna 62. In contrast to the embodiment shown in FIG. 5, antenna 62 of FIG. 6 has its distal tip lying substantially flush with the outer periphery of the cannula's distal tip. Once antenna 62 is exited, electromagnetic waves, exiting from the opening of the cannula's tip, will generally propagate along a path S2 towards the desired electro-conductive region of the balloon's periphery. Since the desired target region of balloon 12 is preferably juxtaposed with a deceased tissue, the latter will be effectively thermally treated. Meanwhile, the rest of the periphery of balloon 12 is minimally thermally affected and, thus, does not damage healthy biological tissues.

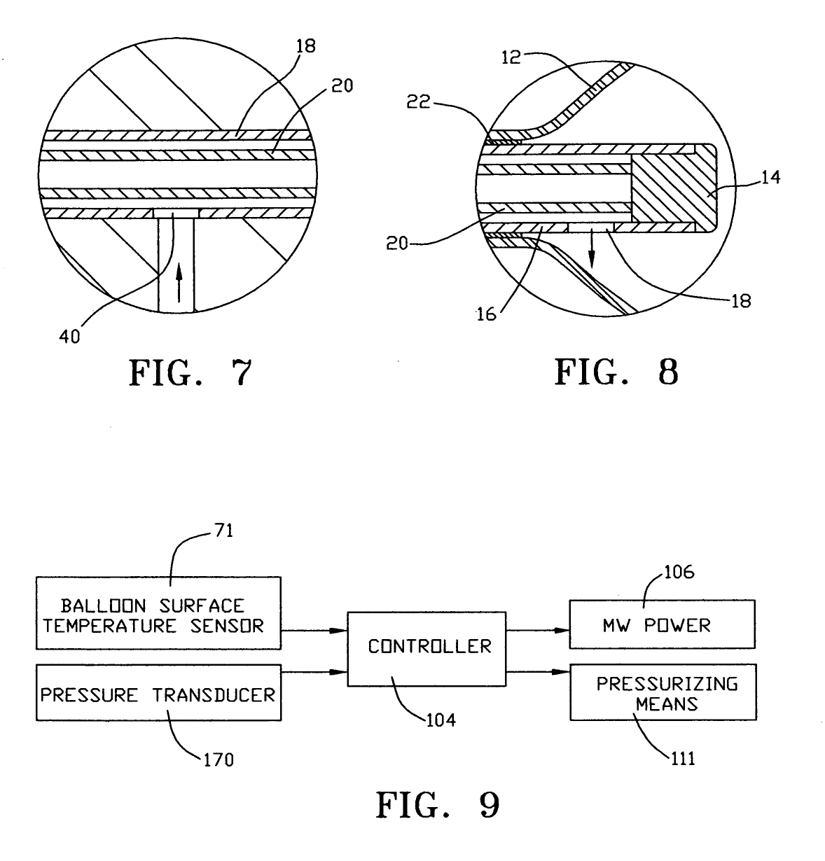

Turning to FIGS. 2, 7 and 8, body 28 is provided with an offset channel 30 which is sealingly coupled to pneumatic supply line 34 by a sealing element 32 so that supply line 34 and channel 30 are in flow communication. The channel 30 extends generally parallel to the longitudinal axis of body 28 and has a distal end extending transversely to the longitudinal axis and opening into an inlet port 40 of body 28 in the vicinity of the distal end of body 28. Upon traversing port 40, fluid is further advanced along body 28 towards an outlet port 18 located within balloon 12, as illustrated in FIG. 4. As the fluid is exiting into balloon 12, the latter expands filling the patient's cavity.

Referring to FIGS. 1 and 9, in operation, the apparatus is inserted into the patient's cavity and the pressurized gas from a fluid pressurizing device 111 is supplied to inflatable balloon 12, which causes the balloon to expand and fill the treated cavity. The required level of the pneumatic pressure is determined by controller 104 and monitored by pressure transducer 110. The microwave generator 106 is then energized to excite antenna 14 through wires 38. The antenna 14 produces waves in the microwave range which are then being absorbed by the wave-absorbing particles of the elastomeric material in the peripheral wall of inflatable balloon 12. The microwave energy absorbed by the wave-absorbing particles is transformed into heat energy, which causes the ablation of the treated tissue. The level of temperature sufficient to cause the ablation and the time required to reach this temperature are determined by the amount of microwave energy produced by the microwave generator and the density of the wave-absorbing particles in the conductive elastomeric material. Generally, the level of the generated microwave energy is selected to reach the maximum ablation temperature in a shortest period of time, in order to reduce the time of treatment and thus prevent or minimize the undesirable heat transfer from treated diseased tissue to neighboring healthy tissue. A temperature sensor 71 is operative to monitor a temperature of the balloon periphery and coupled to controller 104, which, in turn, is operative to control power source 106 so as to maintain the desired temperature. In case of rapture of balloon 12 or a sudden cavity contraction, the pressure inside inflatable balloon 12 may go outside of the range preset in controller 104. In such a case, the pressure transducer 110 provides the feedback of the pressure change to the controller 104 which is operative to shut off pneumatic pressurizing device 111 and microwave generator 106.

The specific features described herein may be used in some embodiments, but not in others, without departure from the spirit and scope of the invention as set forth. Many additional modifications are intended in the foregoing disclosure, and it will be appreciated by those of ordinary skill in the art that in some instances some features of the invention will be employed in the absence of a corresponding use of other features. Furthermore, although operating the inventive apparatus in a microwave range has been disclosed, other RF wave lengths can be successfully utilized within the scope of the invention. The disclosed apparatus can be used in a variety of surgeries including, for example, endometrial ablation. The illustrative examples therefore do not define the metes and bounds of the invention and the legal protection is afforded the appended claims.

1. An apparatus for ablating deceased biological tissues comprising:
a guidable cannula configured to penetrate into a cavity in a body of a patient; and
an inflatable balloon coupled to the cannula and having a peripheral wall, the peripheral wall being made from composite material with a plurality of particles absorbing radio-frequency waves and heatable to a predetermined temperature for ablating the deceased biological tissue.

2. The apparatus of claim 1, further comprising a pneumatic line coupled to the cannula and supplying a gaseous medium for inflating the balloon, and an antenna coupled to the cannula and extending into the inflatable balloon, the antenna being operative to emit the radio-frequency waves in a microwave range propagating through the gaseous medium in the inflatable balloon and absorbed by the plurality of particles.

3. The apparatus of claim 2, wherein the material of the balloon includes silicones impregnated with the particles selected from the group consisting of nickel, nickel-plated graphite, silver-plated aluminum, silver-plated copper, silver-plated nickel, silver-plated glass, pure silver, fluorosilicone, fluorocarbon, and ethylene-propylene terpolymer and a combination thereof.

4. The apparatus of claim 3, wherein the plurality of particles are spaced apart over an entire surface of the peripheral wall of the balloon.

5. The apparatus of claim 3, wherein the plurality of particles are clustered so as to define at least one wave absorbing wall region of the balloon capable of absorbing the radio frequency waves and at least one wave penetrating wall region, the at least wave penetrating region being substantially thermally unaffected by the penetrating radio-frequency waves.

6. The apparatus of claim 5, wherein the balloon is configured to have the at least one or more wave absorbing wall regions configured to oppose the deceased biological tissues upon inserting the balloon into the cavity.

7. The apparatus of claim 2, wherein a distal end of the cannula has a channel configured to receive the antenna and opening into the balloon so that the radio frequency waves propagate towards a wall region of the peripheral wall of the balloon substantially aligned with the channel and heated to temperature to ablate the deceased biological tissue.

8. The apparatus of claim 7, wherein the antenna has a linear body extending between proximal and distal ends thereof and coaxially with a longitudinal axis of the cannula.

9. The apparatus of claim 7, wherein the channel and the antenna have respective distal ends extending transversely to a longitudinal axis of the cannula.

10. The apparatus of claim 9, wherein the distal end of the antenna is spaced inwards from the distal end of the channel.

11. The apparatus of claim 10, wherein the distal end of the antenna and the distal end of the cannula are flush.

12. The apparatus of claim 2, further comprising a power source operative to excite the antenna, a conductive element coupling the power source to the antenna and extending through the body into the cannula, and a source of the pressurized gaseous medium delivered into the balloon along a fluid path through the body and through the cannula.

13. An apparatus for thermal treating of biological tissues comprising:
a guidable cannula configured to penetrate into a cavity in a body of a patient;
an inflatable balloon sealingly coupled to the cannula; and
an antenna coupled to the cannula and terminating in the balloon, the antenna being exitable to emit radio-frequency waves in a microwave range propagating through a gaseous medium in the balloon so as to selectively heat a peripheral wall of the balloon to a temperature sufficient to ablate deceased biological tissues in the cavity.

14. The apparatus of claim 13, further comprising:
a plug closing a proximate end of the cannula,
a proximate isolator mounted in the cannula and spaced from the plug,
a distal isolator spaced from the proximate isolator in the cannula, and
outer and inner radially spaced electrodes extending from the distal and proximal isolators, respectively, within the cannula and having respective distal electrode ends coupled to the antenna.

15. The apparatus of claim 14, further comprising a power source outside the cannula, an electro-conductive element electrically connecting the power source to the outer and inner electrodes to excite the antenna, and a conduit traversed by the gaseous medium and provided in the cannula so that an outlet end of the conduit opens into the cannula, the cannula being configured with a channel in flow communication with the conduit and having an outlet port open into the balloon so that the fluid traversing the outlet port fills the balloon inflatable to urge against an inner surface of the cavity.

16. The apparatus of claim 15, further comprising a pressure transducer in flow communication with the conduit and operative to monitor a pressure of the gaseous medium in the balloon, a temperature transducer operative to monitor a temperature of the peripheral wall of the of the balloon, and a control unit operative to receive output signals from respective pressure and temperature transducers and control an output of the power source and the pressure of the gaseous medium in the balloon.

17. The apparatus of claim 13, wherein the peripheral wall of the balloon is made from microwave penetrating material impregnated with a plurality of radiowave absorbing particles to be heated at the predetermined temperature.

18. The apparatus of claim 13, wherein a distal end of the cannula has a channel configured to receive the antenna and opening into the balloon so that the radio frequency waves propagate towards a wave absorbing wall region of the peripheral wall heated at the predetermined temperature higher than a temperature of regions of the peripheral wall adjacent to the wave absorbing region.

19. The apparatus of claim 18, wherein a distal end of the antenna is spaced inwards from a distal end of the channel.

20. The apparatus of claim 17, wherein the wave penetrating material of the balloon includes silicones, the radiowave absorbing particles being selected from the group consisting of nickel, nickel-plated graphite, silver-plated aluminum, silver-plated copper, silver-plated nickel, silver-plated glass, pure silver, fluorosilicone, fluorocarbon, and ethylene-propylene terpolymer and a combination thereof.
